SEQUENCE LISTING (16 sequences)

SEQ ID NO: 1 - DNA, 11 bp, synthetic construct
  misc_feature 1..11: note = Description of Artificial Sequence: Synthetic oligonucleotide
  source 1..11: mol_type = other DNA
aaaaaattttt

SEQ ID NO: 2 - DNA, 11 bp, synthetic construct
  misc_feature 1..11: note = Description of Artificial Sequence: Synthetic oligonucleotide
  source 1..11: mol_type = other DNA
aaaaatttttt

SEQ ID NO: 3 - DNA, 12 bp, synthetic construct
  misc_feature 1..12: note = Description of Artificial Sequence: Synthetic oligonucleotide
  source 1..12: mol_type = other DNA
aaaaaacttttt

SEQ ID NO: 4 - DNA, 10 bp, synthetic construct
  misc_feature 1..10: note = Description of Artificial Sequence: Synthetic oligonucleotide
  source 1..10: mol_type = other DNA
aaaaactttt

SEQ ID NO: 5 - DNA, 100 bp, synthetic construct
  misc_feature 1..100: note = Description of Artificial Sequence: Synthetic polynucleotide
  source 1..100: mol_type = other DNA
tacgtacgtctgagcatcgatcgatgtacagctacgtacgtctgagcatcgatcgatgtacagctacgtacgtctgagcatcgatcgatgtacagctacg

SEQ ID NO: 6 - DNA, 35 bp, synthetic construct
  misc_feature 1..35: note = Description of Artificial Sequence: Synthetic oligonucleotide
  source 1..35: mol_type = other DNA
aggtgaggccacagtcattgaggccctgcaggccc

SEQ ID NO: 7 - DNA, 35 bp, synthetic construct
  misc_feature 1..35: note = Description of Artificial Sequence: Synthetic oligonucleotide
  source 1..35: mol_type = other DNA
gtgaggtccacagtcattgtaggccctgcaggccc

SEQ ID NO: 8 - DNA, 35 bp, synthetic construct
  misc_feature 1..35: note = Description of Artificial Sequence: Synthetic oligonucleotide
  source 1..35: mol_type = other DNA
aaggtgaggccacagtcattgaggccctgcaggcc

SEQ ID NO: 9 - DNA, 35 bp, synthetic construct
  misc_feature 1..35: note = Description of Artificial Sequence: Synthetic oligonucleotide
  source 1..35: mol_type = other DNA
aaggtgaggccacagtcattgaggccctgcagccc

SEQ ID NO: 10 - DNA, 35 bp, synthetic construct
  misc_feature 1..35: note = Description of Artificial Sequence: Synthetic oligonucleotide
  source 1..35: mol_type = other DNA
gacggcccgagcagtcattgaggccctgcaggccc

SEQ ID NO: 11 - DNA, 36 bp, synthetic construct
  misc_feature 1..36: note = Description of Artificial Sequence: Synthetic oligonucleotide
  source 1..36: mol_type = other DNA
ttgaggccccgcaggccccctccggacgtcaccacg

SEQ ID NO: 12 - DNA, 37 bp, synthetic construct
  misc_feature 1..37: note = Description of Artificial Sequence: Synthetic oligonucleotide
  source 1..37: mol_type = other DNA
attgaggccctgcaggcccctccggacgtcaccacgg

SEQ ID NO: 13 - DNA, 37 bp, synthetic construct
  misc_feature 1..37: note = Description of Artificial Sequence: Synthetic oligonucleotide
  source 1..37: mol_type = other DNA
attgaggccctgcaggcccctccggacgcaccacggt

SEQ ID NO: 14 - DNA, 37 bp, synthetic construct
  misc_feature 1..37: note = Description of Artificial Sequence: Synthetic oligonucleotide
  source 1..37: mol_type = other DNA
attgaggccctgcaggccccctccggacgtcaccacg

SEQ ID NO: 15 - DNA, 37 bp, synthetic construct
  misc_feature 1..37: note = Description of Artificial Sequence: Synthetic oligonucleotide
  source 1..37: mol_type = other DNA
attgaggccctgcaggcccctccgggacgtcaccacg

SEQ ID NO: 16 - DNA, 72 bp, synthetic construct
  misc_feature 1..72: note = Description of Artificial Sequence: Synthetic oligonucleotide
  source 1..72: mol_type = other DNA
cggaaaagacaggaggcagaaggtgaggccacagtctccggacgtcaccacggtggccgtggaggagacaat

(19) United States
(12) Patent Application Publication
(10) Pub. No.: US 2025/0011863 A1
(43) Pub. Date: Jan. 9, 2025

(54) SYSTEMS AND METHODS FOR IDENTIFYING SEQUENCE VARIATION

(71) Applicant: Life Technologies Corporation, Carlsbad, CA (US)

(72) Inventors: Dumitru Brinza, Montara, CA (US); Zheng Zhang, Arcadia, CA (US); Fiona Hyland, San Mateo, CA (US); Rajesh Gottimukkala, Fremont, CA (US)

(21) Appl. No.: 18/769,897

(22) Filed: Jul. 11, 2024

Related U.S. Application Data
(63) Continuation of application No. 16/948,915, filed on Oct. 6, 2020.
Application No. 16/948,915 is a continuation of application No. 15/497,872, filed on Apr. 26, 2017, now abandoned.
Application No. 15/497,872 is a division of application No. 13/890,923, filed on May 9, 2013, now abandoned.
(60) Provisional application No. 61/683,011, filed on Aug. 14, 2012.
(60) Provisional application No. 61/644,771, filed on May 9, 2012.

(51) Int. Cl. C12Q 1/6874; G16B 5/00; G16B 5/20; G16B 30/00; G16B 30/10; G16B 30/20

(52) U.S. Cl. CPC C12Q 1/6874; G16B 5/20; G16B 30/00; G16B 30/10; G16B 5/00; G16B 30/20

(57) ABSTRACT

Systems and method for determining variants can receive mapped reads, align flow space information to a flow space representation of a corresponding portion of the reference. Reads spanning a position with a potential variant can be evaluated in a context specific manner. A list of probable variants can be provided.

### RELATED APPLICATIONS

This application is a continuation of U.S. application Ser. No. 16/948,915 filed Oct. 6, 2020, which is a continuation of U.S. application Ser. No. 15/497,872 filed Apr. 26, 2017 (now abandoned), which is a divisional of U.S. application Ser. No. 13/890,923 filed May 9, 2013 (now abandoned), which claims priority to U.S. application No. 61/683,011filed Aug. 14, 2012, and U.S. application no. 61/644,771 filed May 9, 2012, which disclosures are herein incorporated by reference in their entirety.

### FIELD

The present disclosure generally relates to the field of nucleic acid sequencing including systems and methods for identifying genomic variants using nucleic acid sequencing data.

### INTRODUCTION

Upon completion of the Human Genome Project, one focus of the sequencing industry has shifted to finding higher throughput and/or lower cost nucleic acid sequencing technologies, sometimes referred to as “next generation” sequencing (NGS) technologies. In making sequencing higher throughput and/or less expensive, the goal is to make the technology more accessible. These goals can be reached through the use of sequencing platforms and methods that provide sample preparation for samples of significant complexity, sequencing larger numbers of samples in parallel (for example through use of barcodes and multiplex analysis), and/or processing high volumes of information efficiently and completing the analysis in a timely manner. Various methods, such as, for example, sequencing by synthesis, sequencing by hybridization, and sequencing by ligation are evolving to meet these challenges.

Ultra-high throughput nucleic acid sequencing systems incorporating NGS technologies typically produce a large number of short sequence reads. Sequence processing methods should desirably assemble and/or map a large number of reads quickly and efficiently, such as to minimize use of computational resources. For example, data arising from sequencing of a mammalian genome can result in tens or hundreds of millions of reads that typically need to be assembled before they can be further analyzed to determine their biological, diagnostic and/or therapeutic relevance.

Exemplary applications of NGS technologies include, but are not limited to: genomic variant detection, such as insertions/deletions, copy number variations, single nucleotide polymorphisms, etc., genomic resequencing, gene expression analysis and genomic profiling. Of particular interest are improved systems and methods for detecting insertions and deletions.

From the foregoing it will be appreciated that a need exists for systems and methods that can detect InDel variants using nucleic acid sequencing data.

### BRIEF DESCRIPTION OF DRAWINGS

For a more complete understanding of the principles disclosed herein, and the advantages thereof, reference is now made to the following descriptions taken in conjunction with the accompanying drawings, in which:

FIG. 1 is a block diagram that illustrates an exemplary computer system, in accordance with various embodiments.

FIG. 2 is a schematic diagram of an exemplary system for reconstructing a nucleic acid sequence, in accordance with various embodiments.

FIG. 3 is a flow diagram illustrating an exemplary method of calling variants, in accordance with various embodiments.

FIG. 4 is a schematic diagram of an exemplary variant calling system, in accordance with various embodiments.

FIG. 5 is a flow diagram illustrating an exemplary method of identifying insertions or deletions, in accordance with various embodiments.

FIG. 6 is a flow diagram illustrating an exemplary method of identifying insertions or deletions within di-nucleotide and tri-nucleotide repeat regions, in accordance with various embodiments.

FIG. 7 is a flow diagram illustrating an exemplary method of realigning a read and a target sequence in flow space, in accordance with various embodiments.

FIG. 8 is a flow diagram illustrating an exemplary method of identifying insertions or deletions, in accordance with various embodiments.

FIG. 9 is a flow diagram illustrating an exemplary method of local sequence assembly, in accordance with various embodiments.

FIG. 10 is an exemplary ionogram in accordance with various embodiments.

FIGS. 11 and 12 are illustrations of the relationship between flow space and base space, in accordance with various embodiments.

FIG. 13 illustrates an exemplary alignment of a plurality of reads at a soft clipped region.

It is to be understood that the figures are not necessarily drawn to scale, nor are the objects in the figures necessarily drawn to scale in relationship to one another. The figures are depictions that are intended to bring clarity and understanding to various embodiments of apparatuses, systems, and methods disclosed herein. Wherever possible, the same reference numbers will be used throughout the drawings to refer to the same or like parts. Moreover, it should be appreciated that the drawings are not intended to limit the scope of the present teachings in any way.

### DESCRIPTION OF VARIOUS EMBODIMENTS

Embodiments of systems and methods for detecting variants are described herein, which includes the accompanying Exhibits.

The section headings used herein are for organizational purposes only and are not to be construed as limiting the described subject matter in any way.

In this detailed description of the various embodiments, for purposes of explanation, numerous specific details are set forth to provide a thorough understanding of the embodiments disclosed. One skilled in the art will appreciate, however, that these various embodiments may be practiced with or without these specific details. In other instances, structures and devices are shown in block diagram form. Furthermore, one skilled in the art can readily appreciate that the specific sequences in which methods are presented and performed are illustrative and it is contemplated that the sequences can be varied and still remain within the spirit and scope of the various embodiments disclosed herein.

All literature and similar materials cited in this application, including but not limited to, patents, patent applications, articles, books, treatises, and internet web pages are expressly incorporated by reference in their entirety for any purpose. Unless described otherwise, all technical and scientific terms used herein have a meaning as is commonly understood by one of ordinary skill in the art to which the various embodiments described herein belongs.

It will be appreciated that there is an implied “about” prior to the temperatures, concentrations, times, number of bases, coverage, etc. discussed in the present teachings, such that slight and insubstantial deviations are within the scope of the present teachings. In this application, the use of the singular includes the plural unless specifically stated otherwise. Also, the use of “comprise”, “comprises”, “comprising”, “contain”, “contains”, “containing”, “include”, “includes”, and “including” are not intended to be limiting. It is to be understood that both the foregoing general description and the following detailed description are exemplary and explanatory only and are not restrictive of the present teachings.

Further, unless otherwise required by context, singular terms shall include pluralities and plural terms shall include the singular. Generally, nomenclatures utilized in connection with, and techniques of, cell and tissue culture, molecular biology, and protein and oligo-or polynucleotide chemistry and hybridization described herein are those well known and commonly used in the art. Standard techniques are used, for example, for nucleic acid purification and preparation, chemical analysis, recombinant nucleic acid, and oligonucleotide synthesis. Enzymatic reactions and purification techniques are performed according to manufacturer's specifications or as commonly accomplished in the art or as described herein. The techniques and procedures described herein are generally performed according to conventional methods well known in the art and as described in various general and more specific references that are cited and discussed throughout the instant specification. See, e.g., Sambrook et al., Molecular Cloning: A Laboratory Manual (Third ed., Cold Spring Harbor Laboratory Press, Cold Spring Harbor, N.Y. 2000). The nomenclatures utilized in connection with, and the laboratory procedures and techniques described herein are those well-known and commonly used in the art.

As used herein, “a” or “an” also may refer to “at least one” or “one or more.”

A “system” sets forth a set of components, real or abstract, comprising a whole where each component interacts with or is related to at least one other component within the whole.

A “biomolecule” may refer to any molecule that is produced by a biological organism, including large polymeric molecules such as proteins, polysaccharides, lipids, and nucleic acids (DNA and RNA) as well as small molecules such as primary metabolites, secondary metabolites, and other natural products.

The phrase “next generation sequencing” or NGS refers to sequencing technologies having increased throughput as compared to traditional Sanger- and capillary electrophoresis-based approaches, for example with the ability to generate hundreds of thousands of relatively small sequence reads at a time. Some examples of next generation sequencing techniques include, but are not limited to, sequencing by synthesis, sequencing by ligation, and sequencing by hybridization. More specifically, the Personal Genome Machine (PGM) of Life Technologies Corp. provides massively parallel sequencing with enhanced accuracy. The PGM System and associated workflows, protocols, chemistries, etc. are described in more detail in U.S. Patent Application Publication No. 2009/0127589 and No. 2009/0026082, the entirety of each of these applications being incorporated herein by reference.

The phrase “sequencing run” refers to any step or portion of a sequencing experiment performed to determine some information relating to at least one biomolecule (e.g., nucleic acid molecule).

The phrase “base space” refers to a representation of the sequence of nucleotides. The phrase “flow space” refers to a representation of the incorporation event or non-incorporation event for a particular nucleotide flow. For example, flow space can be a series of zeros and ones representing a nucleotide incorporation event (a one, “1”) or a non-incorporation event (a zero, “0”) for that particular nucleotide flow. It should be understood that zeros and ones are convenient representations of a non-incorporation event and a nucleotide incorporation event; however, any other symbol or designation could be used alternatively to represent and/or identify these events and non-events.

DNA (deoxyribonucleic acid) is a chain of nucleotides consisting of 4 types of nucleotides; A (adenine), T (thymine), C (cytosine), and G (guanine), and that RNA (ribonucleic acid) is comprised of 4 types of nucleotides; A, U (uracil), G, and C. Certain pairs of nucleotides specifically bind to one another in a complementary fashion (called complementary base pairing). That is, adenine (A) pairs with thymine (T) (in the case of RNA, however, adenine (A) pairs with uracil (U)), and cytosine (C) pairs with guanine (G). When a first nucleic acid strand binds to a second nucleic acid strand made up of nucleotides that are complementary to those in the first strand, the two strands bind to form a double strand. As used herein, “nucleic acid sequencing data,” “nucleic acid sequencing information,” “nucleic acid sequence,” “genomic sequence,” “genetic sequence,” or “fragment sequence,” or “nucleic acid sequencing read” denotes any information or data that is indicative of the order of the nucleotide bases (e.g., adenine, guanine, cytosine, and thymine/uracil) in a molecule (e.g., whole genome, whole transcriptome, exome, oligonucleotide, polynucleotide, fragment, etc.) of DNA or RNA. It should be understood that the present teachings contemplate sequence information obtained using all available varieties of techniques, platforms or technologies, including, but not limited to: capillary electrophoresis, microarrays, ligation-based systems, polymerase-based systems, hybridization-based systems, direct or indirect nucleotide identification systems, pyrosequencing, ion- or pH-based detection systems, electronic signature-based systems, etc.

A “polynucleotide”, “nucleic acid”, or “oligonucleotide” refers to a linear polymer of nucleosides (including deoxyribonucleosides, ribonucleosides, or analogs thereof) joined by internucleosidic linkages. Typically, a polynucleotide comprises at least three nucleosides. Usually oligonucleotides range in size from a few monomeric units, e.g. 3-4, to several hundreds of monomeric units. Whenever a polynucleotide such as an oligonucleotide is represented by a sequence of letters, such as “ATGCCTG,” it will be understood that the nucleotides are in 5′->3′ order from left to right and that “A” denotes deoxyadenosine, “C” denotes deoxycytidine, “G” denotes deoxyguanosine, and “T” denotes thymidine, unless otherwise noted. The letters A, C, G, and T may be used to refer to the bases themselves, to nucleosides, or to nucleotides comprising the bases, as is standard in the art.

The techniques of “paired-end,” “pairwise,” “paired tag,” or “mate pair” sequencing are generally known in the art of molecular biology (Siegel A. F. et al., Genomics. 2000, 68:237-246; Roach J. C. et al., Genomics. 1995, 26:345-353). These sequencing techniques provide for the determination of multiple “reads” of sequence information from different regions on a polynucleotide strand. Typically, the distance, such as an insert region or a gap, between the reads or other information regarding a relationship between the reads is known or can be approximated. In some situations, these sequencing techniques provide more information than does sequencing stretches of nucleic acid sequences in a random fashion. With the use of appropriate software tools for the assembly of sequence information (e.g., Millikin S. C. et al., Genome Res. 2003, 13:81-90; Kent, W. J. et al., Genome Res. 2001, 11:1541-8) it is possible to make use of the knowledge that the “paired-end,” “pairwise,” “paired tag” or “mate pair” sequences are not completely random, but are known or anticipated to occur some distance apart and/or to have some other relationship, and are therefore linked or paired with respect to their position within the genome. This information can aid in the assembly of whole nucleic acid sequences into a consensus sequence.

### Computer-Implemented System

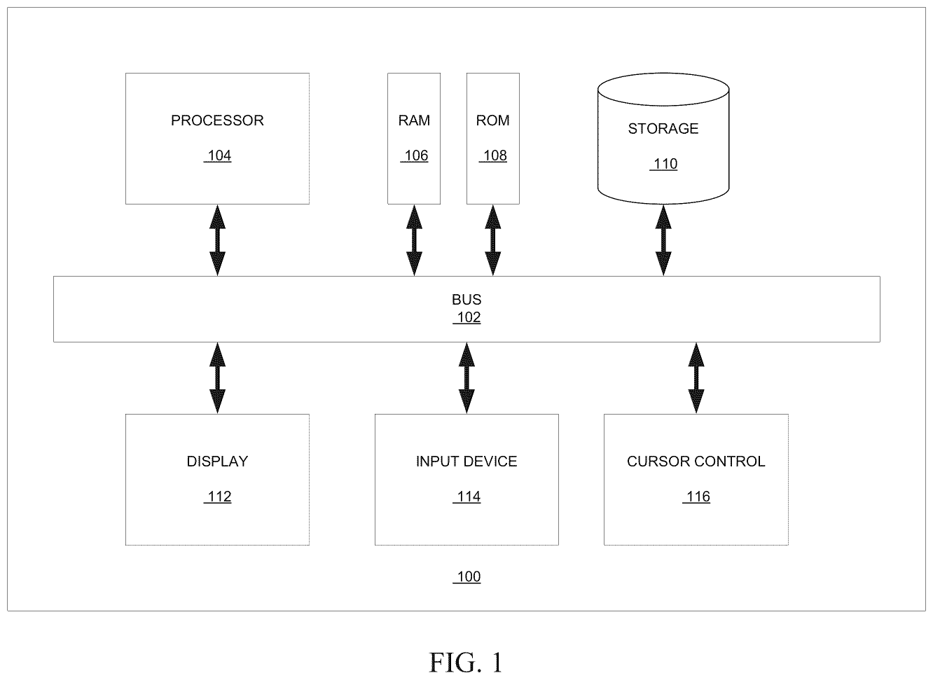

FIG. 1 is a block diagram that illustrates a computer system 100, upon which embodiments of the present teachings may be implemented. In various embodiments, computer system 100 can include a bus 102 or other communication mechanism for communicating information, and a processor 104 coupled with bus 102 for processing information. In various embodiments, computer system 100 can also include a memory 106, which can be a random access memory (RAM) or other dynamic storage device, coupled to bus 102 for determining base calls, and instructions to be executed by processor 104. Memory 106 also can be used for storing temporary variables or other intermediate information during execution of instructions to be executed by processor 104. In various embodiments, computer system 100 can further include a read only memory (ROM) 108 or other static storage device coupled to bus 102 for storing static information and instructions for processor 104. A storage device 110, such as a magnetic disk or optical disk, can be provided and coupled to bus 102 for storing information and instructions.

In various embodiments, computer system 100 can be coupled via bus 102 to a display 112, such as a cathode ray tube (CRT) or liquid crystal display (LCD), for displaying information to a computer user. An input device 114, including alphanumeric and other keys, can be coupled to bus 102 for communicating information and command selections to processor 104. Another type of user input device is a cursor control 116, such as a mouse, a trackball or cursor direction keys for communicating direction information and command selections to processor 104 and for controlling cursor movement on display 112. This input device typically has two degrees of freedom in two axes, a first axis (i.e., x) and a second axis (i.e., y), that allows the device to specify positions in a plane.

A computer system 100 can perform the present teachings. Consistent with certain implementations of the present teachings, results can be provided by computer system 100 in response to processor 104 executing one or more sequences of one or more instructions contained in memory 106. Such instructions can be read into memory 106 from another computer-readable medium, such as storage device 110. Execution of the sequences of instructions contained in memory 106 can cause processor 104 to perform the processes described herein. Alternatively hard-wired circuitry can be used in place of or in combination with software instructions to implement the present teachings. Thus implementations of the present teachings are not limited to any specific combination of hardware circuitry and software.

The term “computer-readable medium” as used herein refers to any media that participates in providing instructions to processor 104 for execution. Such a medium can take many forms, including but not limited to, non-volatile media, volatile media, and transmission media. Examples of non-volatile media can include, but are not limited to, optical or magnetic disks, such as storage device 110. Examples of volatile media can include, but are not limited to, dynamic memory, such as memory 106. Examples of transmission media can include, but are not limited to, coaxial cables, copper wire, and fiber optics, including the wires that comprise bus 102.

Common forms of computer-readable media include, for example, a floppy disk, a flexible disk, hard disk, magnetic tape, or any other magnetic medium, a CD-ROM, any other optical medium, punch cards, paper tape, any other physical medium with patterns of holes, a RAM, PROM, and EPROM, a FLASH-EPROM, any other memory chip or cartridge, or any other tangible medium from which a computer can read.

In accordance with various embodiments, instructions configured to be executed by a processor to perform a method are stored on a computer-readable medium. The computer-readable medium can be a device that stores digital information. For example, a computer-readable medium includes a compact disc read-only memory (CD-ROM) as is known in the art for storing software. The computer-readable medium is accessed by a processor suitable for executing instructions configured to be executed.

### Nucleic Acid Sequencing Platforms

Nucleic acid sequence data can be generated using various techniques, platforms or technologies, including, but not limited to: capillary electrophoresis, microarrays, ligation-based systems, polymerase-based systems, hybridization-based systems, direct or indirect nucleotide identification systems, pyrosequencing, ion- or pH-based detection systems, electronic signature-based systems, etc.

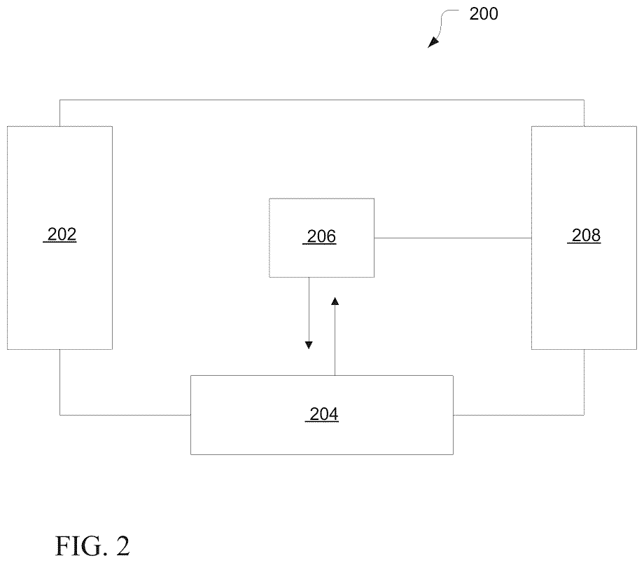

Various embodiments of nucleic acid sequencing platforms, such as a nucleic acid sequencer, can include components as displayed in the block diagram of FIG. 2. According to various embodiments, sequencing instrument 200 can include a fluidic delivery and control unit 202, a sample processing unit 204, a signal detection unit 206, and a data acquisition, analysis and control unit 208. Various embodiments of instrumentation, reagents, libraries and methods used for next generation sequencing are described in U.S. Patent Application Publication No. 2009/0127589 and No. 2009/0026082 are incorporated herein by reference. Various embodiments of instrument 200 can provide for automated sequencing that can be used to gather sequence information from a plurality of sequences in parallel, such as substantially simultaneously.

In various embodiments, the fluidics delivery and control unit 202 can include reagent delivery system. The reagent delivery system can include a reagent reservoir for the storage of various reagents. The reagents can include RNA-based primers, forward/reverse DNA primers, oligonucleotide mixtures for ligation sequencing, nucleotide mixtures for sequencing-by-synthesis, optional ECC oligonucleotide mixtures, buffers, wash reagents, blocking reagent, stripping reagents, and the like. Additionally, the reagent delivery system can include a pipetting system or a continuous flow system which connects the sample processing unit with the reagent reservoir.

In various embodiments, the sample processing unit 204 can include a sample chamber, such as flow cell, a substrate, a micro-array, a multi-well tray, or the like. The sample processing unit 204 can include multiple lanes, multiple channels, multiple wells, or other means of processing multiple sample sets substantially simultaneously. Additionally, the sample processing unit can include multiple sample chambers to enable processing of multiple runs simultaneously. In particular embodiments, the system can perform signal detection on one sample chamber while substantially simultaneously processing another sample chamber. Additionally, the sample processing unit can include an automation system for moving or manipulating the sample chamber.

In various embodiments, the signal detection unit 206 can include an imaging or detection sensor. For example, the imaging or detection sensor can include a CCD, a CMOS, an ion or chemical sensor, such as an ion sensitive layer overlying a CMOS or FET, a current or voltage detector, or the like. The signal detection unit 206 can include an excitation system to cause a probe, such as a fluorescent dye, to emit a signal. The excitation system can include an illumination source, such as arc lamp, a laser, a light emitting diode (LED), or the like. In particular embodiments, the signal detection unit 206 can include optics for the transmission of light from an illumination source to the sample or from the sample to the imaging or detection sensor. Alternatively, the signal detection unit 206 may provide for electronic or non-photon based methods for detection and consequently not include an illumination source. In various embodiments, electronic-based signal detection may occur when a detectable signal or species is produced during a sequencing reaction. For example, a signal can be produced by the interaction of a released byproduct or moiety, such as a released ion, such as a hydrogen ion, interacting with an ion or chemical sensitive layer. In other embodiments a detectable signal may arise as a result of an enzymatic cascade such as used in pyrosequencing (see, for example, U.S. Patent Application Publication No. 2009/0325145, the entirety of which being incorporated herein by reference) where pyrophosphate is generated through base incorporation by a polymerase which further reacts with ATP sulfurylase to generate ATP in the presence of adenosine 5′ phosphosulfate wherein the ATP generated may be consumed in a luciferase mediated reaction to generate a chemiluminescent signal. In another example, changes in an electrical current can be detected as a nucleic acid passes through a nanopore without the need for an illumination source.

In various embodiments, a data acquisition analysis and control unit 208 can monitor various system parameters. The system parameters can include temperature of various portions of instrument 200, such as sample processing unit or reagent reservoirs, volumes of various reagents, the status of various system subcomponents, such as a manipulator, a stepper motor, a pump, or the like, or any combination thereof.

It will be appreciated by one skilled in the art that various embodiments of instrument 200 can be used to practice variety of sequencing methods including ligation-based methods, sequencing by synthesis, single molecule methods, nanopore sequencing, and other sequencing techniques.

In various embodiments, the sequencing instrument 200 can determine the sequence of a nucleic acid, such as a polynucleotide or an oligonucleotide. The nucleic acid can include DNA or RNA, and can be single stranded, such as ssDNA and RNA, or double stranded, such as dsDNA or a RNA/cDNA pair. In various embodiments, the nucleic acid can include or be derived from a fragment library, a mate pair library, a ChIP fragment, or the like. In particular embodiments, the sequencing instrument 200 can obtain the sequence information from a single nucleic acid molecule or from a group of substantially identical nucleic acid molecules.

In various embodiments, sequencing instrument 200 can output nucleic acid sequencing read data in a variety of different output data file types/formats, including, but not limited to: *.fasta, *.csfasta, *seq.txt, *qseq.txt, *.fastq, *.sff, *prb.txt, *.sms, *srs and/or *.qv.

### Adaptor-Joining Methods:

In some embodiments, the present teachings are directed to methods for preparing a library of polynucleotide constructs which can include an adaptor-joining step. In some embodiments, a plurality of polynucleotide fragments can include at least two polynucleotide fragments that are joined to one or more nucleic acid adaptors by hybridization (e.g., with or without a primer extension reaction) or enzymatic ligation (e.g., a ligase reaction) to generate adaptor-fragment constructs. In some embodiments, one end or both ends of polynucleotide fragments can be joined to at least one type of adaptor. One or both ends of a polynucleotide fragment can be joined to at least one nucleic acid adaptor, including barcoded adaptors, sequencing primer adaptors, amplification primer adaptors, universal adaptors, blocking oligonucleotide adaptors and/or others.

In some embodiments, an adaptor can include nucleotide sequences that are complementary to sequencing primers (e.g., P1, P2 and/or A), amplification primers, universal sequences and/or barcode sequences. For example, released mate pair constructs can be joined at each end to a different sequencing adaptor to prepare a nucleic acid library for sequencing with SOLiD™ sequencing reactions (WO 2006/084131) or sequencing with ion-sensitive sequencing reactions (e.g., Ion Torrent PGM™ and Proton™ sequencers from Life Technologies Corporation, see for example U.S. Patent Publication Nos. 2010/0301398, 2010/0300895, 2010/0300559, 2010/0197507, 2010/0137143, 2009/0127589; and 2009/0026082, which are incorporated by reference in their entireties).

### Barcoded Adaptor Sequences

In some embodiments, the present teachings are directed to methods for preparing a library of polynucleotide constructs which can include joining at least one end of a plurality of polynucleotide fragments to an adaptor having a barcode sequence. A barcode sequence can be a selected sequence of nucleotide bases (e.g. adenine, guanine, cytosine, thymine, uracil, inosine, or analogs thereof) in the polynucleotide strand that serves to identify the polynucleotide strand and/or distinguish it from other polynucleotide strands (e.g. those containing a different target sequence of interest). In some embodiments, a barcode adaptor can include a unique identification sequence (e.g., barcode sequence). A barcode sequence can be used for various purposes, such as tracking, sorting, and/or identifying the samples.

Because different barcode sequences can be associated with different polynucleotide strands, these barcode sequences may be useful in multiplexed sequencing of different samples. In some embodiments, a barcode adaptor can be used for constructing multiplex nucleic acid libraries. In some embodiments, one or more barcode sequences can allow identification of a particular adaptor among a mixture of different adaptors having different barcodes sequences. For example, a mixture can include 2, 3, 4, 5, 6, 7-10, 10-50, 50-100, 100-200, 200-500, 500-1000, or more different adaptors having unique barcode sequences. Examples of various adaptors having barcode sequences can be found in PCT/US2011/054053 which is incorporated by reference in its entirety.

In various high throughput DNA sequencing technologies (such as sequencing-by-synthesis) it is desirable to permit sequencing of different samples that are pooled together for simultaneous analysis (sometimes referred to as multiplexed sequencing).

When carrying out multiplexed sequencing, it is generally desirable to identify the origin of each sample, and this may require that the sequencing data be deconvolved for each sample. In particular, it can be desirable to uniquely identify the source of the sequence data derived from a multiplex sample (for example, to identify a particular nucleic acid species associated with different sample populations). One approach to facilitate sample identification is the use of unique nucleic acid identifier sequences (barcode adaptors) that are embedded within the sample construct so that sequencing data can be correctly identified or associated with its source sample.

### Flow Space

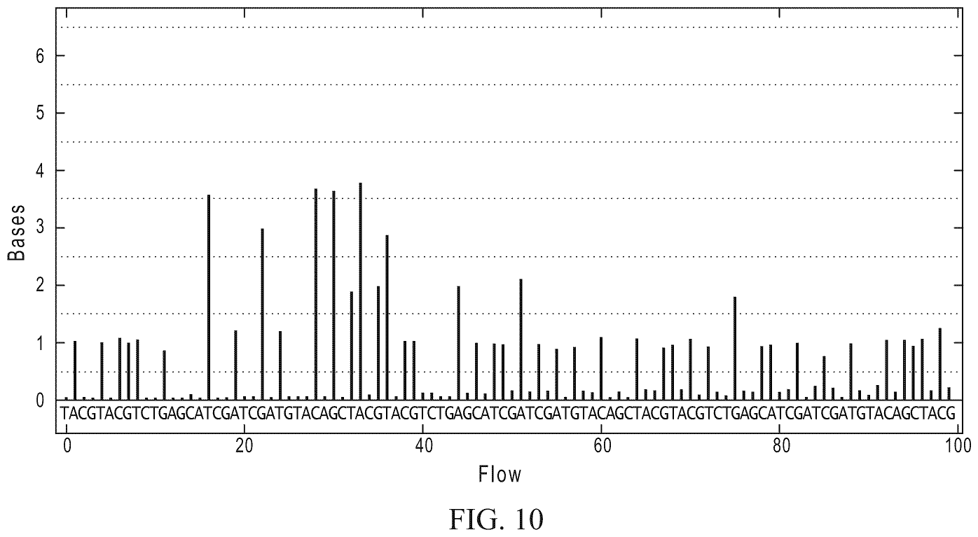

FIG. 10 shows an exemplary ionogram representation of signals from which base calls may be made. In this example, the x-axis shows the nucleotide that is flowed and the corresponding number of nucleotide incorporations may be estimated by rounding to the nearest integer shown in the y-axis, for example. Signals used to make base calls and determine a flowspace vector may be from any suitable point in the acquisition or processing of the data signals received from sequencing operations. For example, the signals may be raw acquisition data or data having been processed, such as, e.g., by background filtering, normalization, correction for signal decay, and/or correction for phase errors or effects, etc. The base calls may be made by analyzing any suitable signal characteristics (e.g., signal amplitude or intensity).

In various embodiments, output signals due to nucleotide incorporation may be processed in various way to improve their quality and/or signal-to-noise ratio, which may include performing or implementing one or more of the teachings disclosed in Rearick et al., U.S. patent application Ser. No. 13/339,846, filed Dec. 29, 2011, and in Hubbell, U.S. patent application Ser. No. 13/339,753, filed Dec. 29, 2011, which are all incorporated by reference herein in their entirety.

In various embodiments, output signals due to nucleotide incorporation may be further processed, given knowledge of what nucleotide species were flowed and in what order to obtain such signals, to make base calls for the flows and compile consecutive base calls associated with a sample nucleic acid template into a read. A base call refers to a particular nucleotide identification (e.g., dATP (“A”), dCTP (“C”), dGTP (“G”), or dTTP (“T”)). Base calling may include performing one or more signal normalizations, signal phase and signal droop (e.g, enzyme efficiency loss) estimations, and signal corrections, and may identify or estimate base calls for each flow for each defined space. Base calling may include performing or implementing one or more of the teachings disclosed in Davey et al., U.S. patent application Ser. No. 13/283,320, filed Oct. 27, 2011, which is incorporated by reference herein in its entirety. Other aspects of signal processing and base calling may include performing or implementing one or more of the teachings disclosed in Davey et al., U.S. patent application Ser. No. 13/340,490, filed on Dec. 29, 2011, and Sikora et al., U.S. patent application Ser. No. 13/588,408, filed on Aug. 17, 2012, which are all incorporated by reference herein in their entirety.

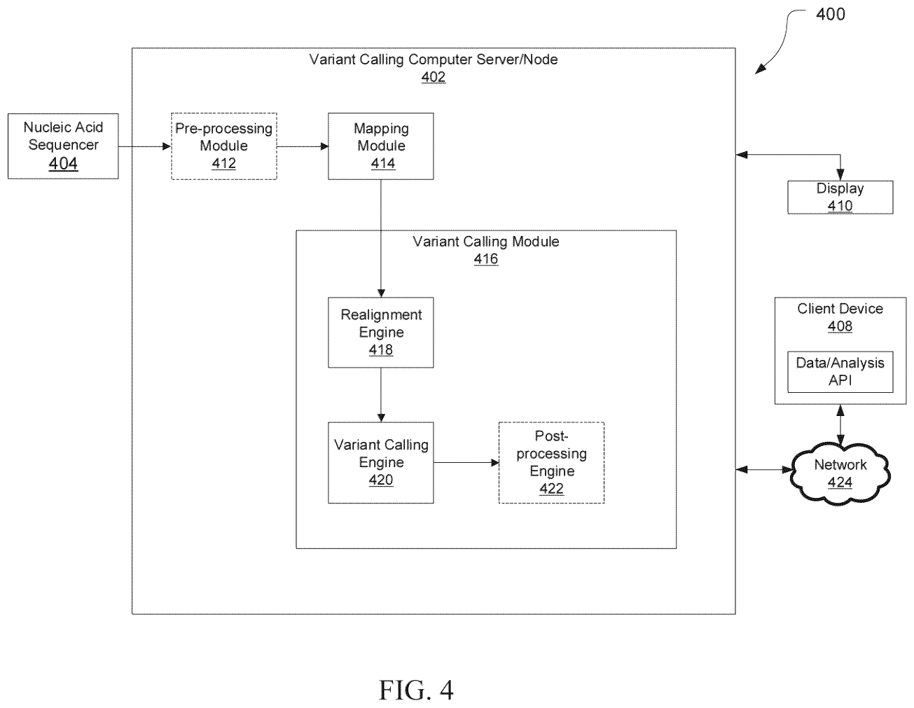

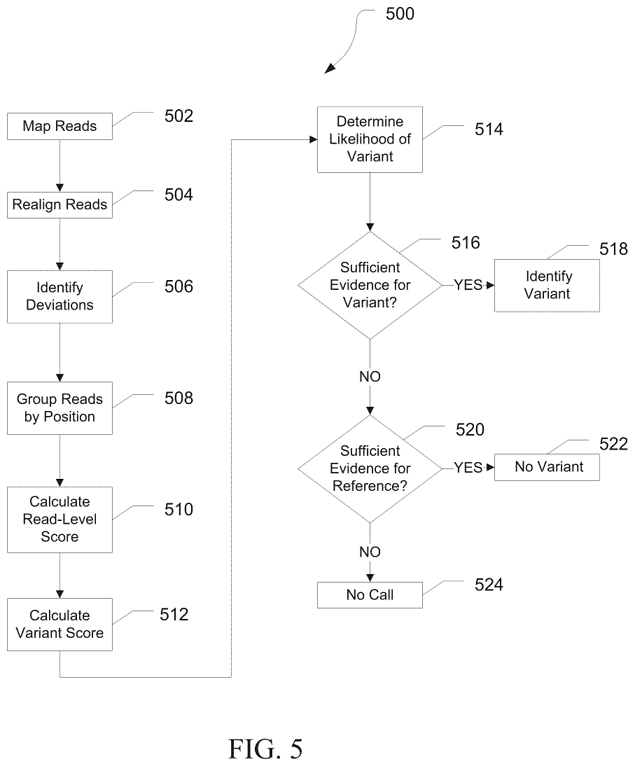

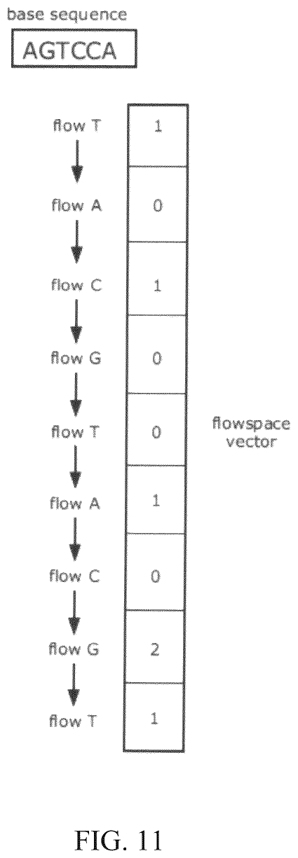

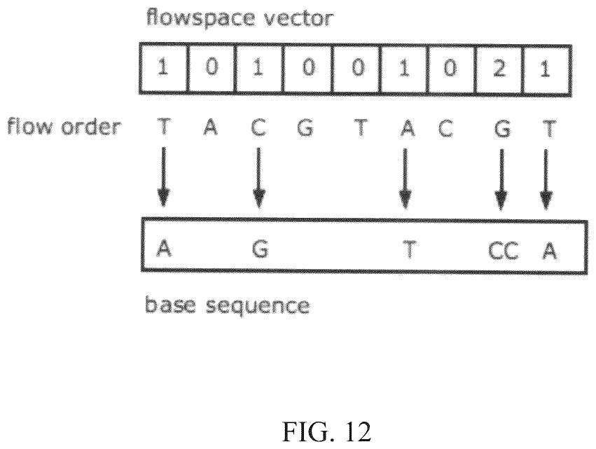

FIGS. 11 and 12 demonstrate a relationship between a base space sequence and a flowspace vector. A series of signals representative of a number of incorporations or lack thereof (e.g., 0-mer, 1-mer, 2-mer, etc.) produced by a series of nucleotide flows may be referred to as a flowspace vector or sequence, as opposed to a base space sequence, which is simply the order of identified nucleotide bases in a nucleic acid of interest. The flowspace vector may be produced using any suitable nucleotide flow ordering, including a predetermined ordering based on a cyclical, repeating pattern of consecutive repeats of a predetermined reagent flow ordering, based on a random reagent flow ordering, or based on an ordering comprising in whole or in part a phase-protecting reagent flow ordering as described in Hubbell et al., U.S. patent application Ser. No. 13/440,849, filed Apr. 5, 2012, or some combination thereof. In FIGS. 4 and 5, an exemplary base space sequence AGTCCA is subjected to sequencing operations using a cyclical flow ordering of TACG (that is, a T nucleotide flow, followed by an A nucleotide flow, followed by a C nucleotide flow, followed by a G nucleotide flow, and this 4-flow ordering is then repeated cyclically). The flows result in a series of signals having an amplitude (e.g., signal intensity) related to the number of nucleotide incorporations (e.g., 0-mer, 1-mer, 2-mer, etc.). This series of signals generates the flowspace vector 101001021. As shown in FIG. 11, the base space sequence AGTCCA may be translated to a flowspace vector 101001021 under a cyclical flow ordering of TACG. The flowspace vector may change if the flow ordering is changed. As shown in FIG. 12, the flowspace vector may be mapped back to the base space sequence associated with the sample.

### System and Methods for Identifying Sequence Variation

Identification of sequence variants including single nucleotide polymorphism (SNPs), insertions, and deletions is an important application of next generation sequencing technologies. In various embodiments, the approach/technology implemented during sequencing can influence the accuracy of variant identification. Likewise, the analytical approach used during sequence resolution and alignment can affect the overall quality of the data. For example, in certain embodiments, base space alignment methodologies can misplace or miscall insertions or deletions in the alignment of flow space reads generated using sequencing by synthesis platforms such as the Ion Torrent PGM.

Example 1: (an example that may occur with increased frequency)

AAAAAATTTTT ← reference
AAAAATTTTTT ← read1
AAAAAATTTTT ← read2
AAAAATTTTTT ← read3
AAAAAATTTTT ← read4

Example 2: (another miss-aligned example)

AAAAAACTTTTT ← reference
AAAAAC--TTTT ← read1
AAAAAACTTTTT ← read2
AAAAAC--TTTT ← read3
AAAAAACTTTTT ← read4

In the examples above the more likely alignment (explanation) for reads 1 and 3 may be as follows:

AAAAAA-TTTTT ← reference
AAAAA-TTTTTT ← read1
AAAAA-TTTTTT ← read3

In various embodiments, although the alignment above may be more likely to be true, it is not necessarily always the correct one. For example, an A→T SNP at the middle position as indicated may not be as rare as expected. Using flow space alignment and pileup to select the above alignments, overlooking or misidentifying such types of alignments may occur. In various instances such as the two alignments shown above two forms (mismatch vs undercall+overcall) may be statistically in the same order of magnitude. In such instances, it may be difficult or impractical for an automated sequence or fragment alignment routine to select or identify the most accurate or true candidate. For example, the likelihood of a mismatch occurring may be approximately 0.5%, and the chance of undercall followed by overcall might be large.

From the foregoing discussion it will be appreciated that during sequence analysis a portion of the alignment may be of lower quality. It is therefore not uncommon that the selected analysis path may lead to an incorrect or lower quality result.

In many cases, however, irrespective of the sequence alignment algorithm used for poorly aligned base reads, it may be expected that the bases are not far from their correct positioning/alignments. This can be observed for single bases as well as multiple bases in an exemplary read.

Using an instrument capable of generating flowspace type sequence data such as the PGM an application may be configured to call indels with a score based on a flow space realignment performed on base space alignment reads. In various embodiments, the flowspace representation may allow for better determination of variants by having different signature between simple intensity differences. Such differences may be part of the systematic error profile of the sequencing system and reflect actual indel variants. In a more detailed example, a sequence deviation in a read that has only a single marginal flow intensity change supporting a variant may be considered as weak evidence, while one that would require some change in the flow order or multiple strong intensity changes, may be stronger evidence of a variant.

In various embodiments, analysis software may be designed and configured to detect or register multi-or non-positional indel events, by representing deviations as sequences of detected differences between the read query and the target reference in a flow space alignment. From this alignment, calculations of sequence deviations, on a read by read basis may be performed, the deviations found in each read, standardizing the representation of the deviations by merging adjacent deviations together and representing them by a position (for example leftmost).

The analysis approach may then group the variants of multiple reads together by position and produces a score based on flow space alignment characteristics. Alignment characteristics for the score may include intensity differences, missing flow bases, and added flow bases, some or all of which may be compared with what would be expected from a reference target sequence. In certain cases, an additional characteristic may be considered relating to how close the flow(s) that specify the sequence deviation are to either the start or end flow for the read. Such a strategy may help make the score representative of the likelihood of the indel event directly without the need for heuristic filtering. Heuristic knowledge may be considered important, however, and that knowledge, as it is amassed, may provide motivation for greater refinement of the scoring method.

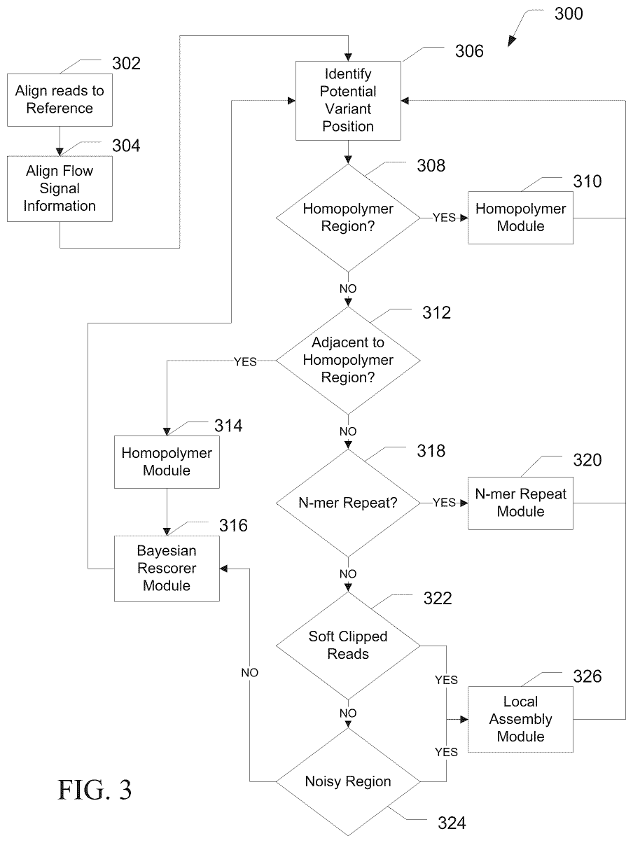

FIG. 3 is an exemplary flow diagram showing a method 300 for identifying indel variants in nucleic acid sequence reads, in accordance with various embodiments.

At 302, reads can be mapped to a reference genome. Various algorithms are known in the art for mapping reads to a reference genome. In particular embodiments, the mapping to the reference genome can be performed in base space after the reads are converted from flow space to base space.

At 304, the mapped reads can be realigned to the reference sequence in flow space. In particular embodiments, the portion of the reference sequence to which a read is mapped can be converted into flow space based upon the flow order and the sequence. The flow space representation of the reference can be aligned to the flow space representation for the read. Flow space realignment may include performing or implementing one or more of the teachings disclosed in Homer, U.S. patent application Ser. No. 13/363,697, filed Feb. 1, 2012, which is incorporated by reference herein in its entirety. The aligned flow space representations provide allow for distinguishing sequencing errors from actual variants on the bases of a flow space signature, fingerprint, or pattern.

At 306, positions where at least some of the reads differ from the reference can be identified as potential variant positions. In various embodiments, some positions can be identified as potential variant positions due to sequencing errors in one or more reads, even though the sample may not have a variant at that position.

At 308, it can be determined if the potential variant position is within a homopolymer region, such as a variation of the length of the homopolymer, such as an apparent deletion of a T from a series of Ts, for example, ATTTTTC where the reference is ATTTTTTC. At 310, when the potential variant position is within a homopolymer region, reads spanning the homopolymer region can be sent to the homopolymer module to determine if there is a variation within the homopolymer region, and, at 306, another potential variant position can be identified.

At 312, when the potential variant position is not within a homopolymer region, it can be determined if the potential variant position is adjacent to a homopolymer region, such as an apparent insertion of an A after a series of Ts, for example, TTTTTAC where the reference is TTTTTC. At 314, when the potential variant position is adjacent to a homopolymer region, reads spanning the homopolymer region and the potential variant position can be sent to the homopolymer module to determine the length of the homopolymer region, and, at 316, a Bayesian rescorer module can evaluate the potential variant position. At 306, another potential variant position can be identified. Bayesian rescorer module can score the potential variant position in accordance with one or more of the teachings disclosed in Hyland et al., U.S. patent application Ser. No. 13/623,709, filed Feb. Sep. 20, 2012, which is incorporated by reference herein in its entirety.

At 318, when the potential variant position is not adjacent to a homopolymer position, it can be determined if the potential variant position is within an n-mer repeat region, such as an apparent deletion of a T from a series of AT repeats, for example GATAATC where the reference is GATATATC. The n-mer repeat region can include a sequence having multiple adjacent copies of a short repeat sequence, such as a di-nucleotide repeat having a repeating sequence of two nucleotides, a tri-nucleotide repeat having a repeating sequence of three nucleotides, or even repeating sequences of four, five, or six nucleotides. At 320, when the potential variant position is within a n-mer repeat region, reads spanning the n-mer repeat region can be sent to the n-mer repeat region module to determine the length of the n-mer repeat region, and, at 306, another potential variant position can be identified.

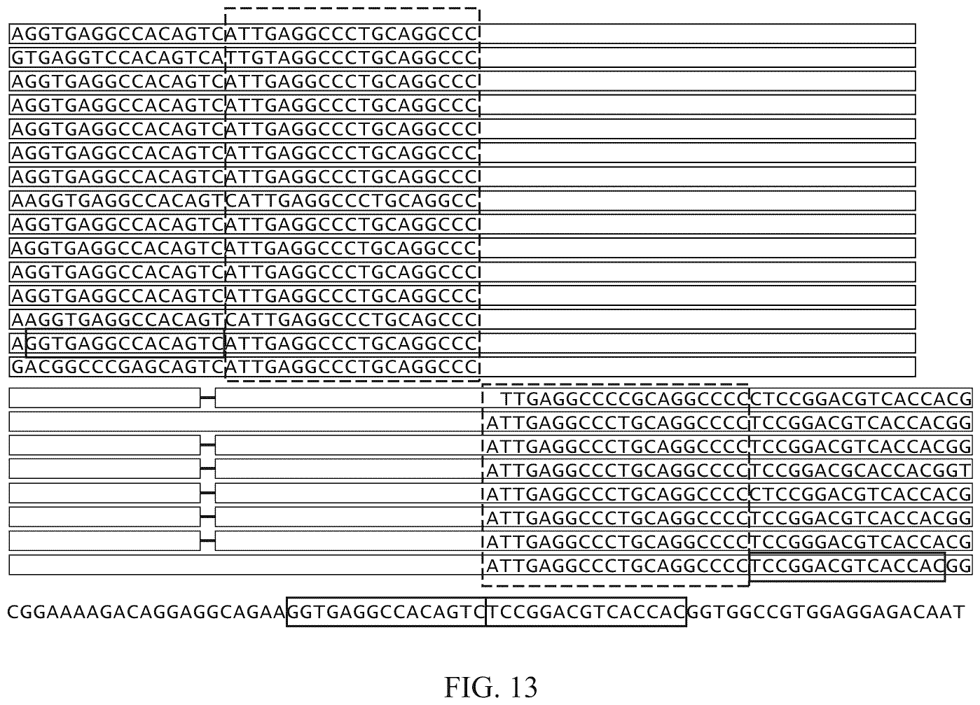

At 322, when the potential variant position is not within a n-mer repeat region, it can be determined if the potential variant position is within a soft clipped region. A soft clipped region is a region where reads spanning the region are partially mapped and partially unmapped. This may be indicative of a read that spans an insertion or deletion. In various embodiments, a portion of the regions may be partially mapped to the left of the soft clipped region and another portion of the reads may be partially mapped to the right of the soft clipped regions. FIG. 13 illustrates an example of a soft clipped region. At 322, when the potential variant position is not within a soft clipped region, it can be determined if the potential variant position is within a noisy region. A noisy region can be a region where there appears to be a large number of potential variants.

At 324, when the potential variant position is either in a soft clipped region or a noisy region, the reads spanning the potential variant position can be sent to a local assembly module 326 to assemble the reads and determine the sequence that spans the soft clipped region or the noisy region, and, at 306, another potential variant position can be identified.

Alternatively, when the potential variant position is not in a soft clipped region or a noisy region, reads spanning the potential variant position can be sent to the Bayesian Rescorer Module 316 to evaluate the evidence for the variant, and, at 306, another potential variant position can be identified.

FIG. 4 is a schematic diagram of a system for identifying variants, in accordance with various embodiments.

As depicted herein, variant analysis system 400 can include a nucleic acid sequence analysis device 404 (e.g., nucleic acid sequencer, real-time/digital/quantitative PCR instrument, microarray scanner, etc.), an analytics computing server/node/device 402, and a display 410 and/or a client device terminal 408.

In various embodiments, the analytics computing sever/node/device 402 can be communicatively connected to the nucleic acid sequence analysis device 404, and client device terminal 408 via a network connection 424 that can be either a “hardwired” physical network connection (e.g., Internet, LAN, WAN, VPN, etc.) or a wireless network connection (e.g., Wi-Fi, WLAN, etc.).

In various embodiments, the analytics computing device/server/node 402 can be a workstation, mainframe computer, distributed computing node (part of a “cloud computing” or distributed networking system), personal computer, mobile device, etc. In various embodiments, the nucleic acid sequence analysis device 404 can be a nucleic acid sequencer, real-time/digital/quantitative PCR instrument, microarray scanner, etc. It should be understood, however, that the nucleic acid sequence analysis device 404 can essentially be any type of instrument that can generate nucleic acid sequence data from samples obtained from an individual.

The analytics computing server/node/device 402 can be configured to host an optional pre-processing module 412, a mapping module 414, and a variant calling module 416.

Pre-processing module 412 can be configured to receive from the nucleic acid sequence analysis device 404 and perform processing steps, such as conversion from f space to base space or from flow space to base space, determining call quality values, preparing the read data for use by the mapping module 414, and the like.

The mapping module 414 can be configured to align (i.e., map) a nucleic acid sequence read to a reference sequence. Generally, the length of the sequence read is substantially less than the length of the reference sequence. In reference sequence mapping/alignment, sequence reads are assembled against an existing backbone sequence (e.g., reference sequence, etc.) to build a sequence that is similar but not necessarily identical to the backbone sequence. Once a backbone sequence is found for an organism, comparative sequencing or re-sequencing can be used to characterize the genetic diversity within the organism's species or between closely related species. In various embodiments, the reference sequence can be a whole/partial genome, whole/partial exome, etc.

In various embodiments, the sequence read and reference sequence can be represented as a sequence of nucleotide base symbols in base space. In various embodiments, the sequence read and reference sequence can be represented as one or more colors in color space. In various embodiments, the sequence read and reference sequence can be represented as nucleotide base symbols with signal or numerical quantitation components in flow space.

In various embodiments, the alignment of the sequence fragment and reference sequence can include a limited number of mismatches between the bases that comprise the sequence fragment and the bases that comprise the reference sequence. Generally, the sequence fragment can be aligned to a portion of the reference sequence in order to minimize the number of mismatches between the sequence fragment and the reference sequence.

The variant calling module 416 can include a realignment engine 418, a variant calling engine 420, and an optional post processing engine 422. In various embodiments, variant calling module 416 can be in communications with the mapping module 414. That is, the variant calling module 416 can request and receive data and information (through, e.g., data streams, data files, text files, etc.) from mapping module 414. In various embodiments, the variant calling module 416 can be configured to communicate variants called for a sample genome as a *.vcf, *.gff, or *.hdf data file. It should be understood, however, that the called variants can be communicated using any file format as long as the called variant information can be parsed and/or extracted for later processing/analysis.

The realignment engine 418 can be configured to receive mapped reads from the mapping module 414, realign the mapped reads in flow space, and provide the flow space alignments to the variant calling engine 420.

The variant calling engine 420 can be configured to receive flow space alignments from the realignment engine 418 and analyze the flow space alignments to detect and call (i.e., identify) one or more genomic indel variants within the reads.

Post processing engine 422 can be configured to receive the variants identified by the variant calling engine 420 and perform additional processing steps, such as conversion from flow space to base space, filtering adjacent variants, and formatting the variant data for display on display 410 or use by client device 408. Examples of filters that the post-processing engine 422 may apply include a minimum score threshold, a minimum number of reads including the variant, a minimum frequency of reads including the variant, a minimum mapping quality, a strand probability, and region filtering.

Client device 408 can be a thin client or thick client computing device. In various embodiments, client terminal 408 can have a web browser (e.g., INTERNET EXPLORER™, FIREFOX™, SAFARI™, etc) that can be used to communicate information to and/or control the operation of the pre-processing module 412, mapping module 414, realignment engine 418, variant calling engine 420, and post processing engine 422 using a browser to control their function. For example, the client terminal 408 can be used to configure the operating parameters (e.g., match scoring parameters, annotations parameters, filtering parameters, data security and retention parameters, etc.) of the various modules, depending on the requirements of the particular application. Similarly, client terminal 408 can also be configured to display the results of the analysis performed by the variant calling module 416 and the nucleic acid sequencer 404.

It should be understood that the various data stores disclosed as part of system 400 can represent hardware-based storage devices (e.g., hard drive, flash memory, RAM, ROM, network attached storage, etc.) or instantiations of a database stored on a standalone or networked computing device(s).

It should also be appreciated that the various data stores and modules/engines shown as being part of the system 400 can be combined or collapsed into a single module/engine/data store, depending on the requirements of the particular application or system architecture. Moreover, in various embodiments, the system 400 can comprise additional modules, engines, components or data stores as needed by the particular application or system architecture.

In various embodiments, the system 400 can be configured to process the nucleic acid reads in color space. In various embodiments, system 400 can be configured to process the nucleic acid reads in base space. In various embodiments, system 400 can be configured to process the nucleic acid sequence reads in flow space. It should be understood, however, that the system 400 disclosed herein can process or analyze nucleic acid sequence data in any schema or format as long as the schema or format can convey the base identity and position of the nucleic acid sequence.

FIG. 5 is an exemplary flow diagram showing a method 500 for identifying variants in nucleic acid sequence reads, in accordance with various embodiments. Using a flow based representation to combine simple intensity differences and flow order differences into a single model can result in improved variant calling. In more detail, a sequence deviation in a read that had only a single marginal flow intensity change supporting a variant can provide weak evidence of a variant, whereas a deviation that required a change in flow order or multiple strong intensity changes can provide strong evidence of a variant.

At 502, reads can be mapped to a reference genome. Various algorithms are known in the art for mapping reads to a reference genome. In particular embodiments, the mapping to the reference genome can be performed in base space after the reads are converted from flow space to base space.

At 504, the mapped reads can be realigned to the reference sequence in flow space. In particular embodiments, the portion of the reference sequence to which a read is mapped can be converted into flow space based upon the flow order and the sequence. The flow space representation of the reference can be aligned to the flow space for the read. At 506, deviations in the aligned flow space can be identified on a read-by-read basis. A standardized representation of the deviations can be generated by merging adjacent deviations and representing them in the leftmost position. At 508, variants of multiple reads can be grouped together.

At 510, a read score can be calculated on per-read and per-variant basis. The per-read, per-variant score can be based on flow-space alignment characteristics, such as intensity differences, missing flow bases, and added flow bases as compared to the flow space representation of the reference. Additionally, the per-read, per-variant score can be further based on the location of the variant within the read, such as a distance from the start of the read or a distance from the end of a read. At 512, a variant score can be calculated on a per-variant basis based on the read scores of the reads that span the variant position.

At 514, the likelihood of a variant can be determined. Statistical modeling can be used to determine the probability of a variant for positions containing evidence of a variant. At 516, it can be determined if there is sufficient evidence for the variant at a position, such as a p-value for the variant being below a predefined threshold. When there is sufficient evidence for the variant at the position, the variant can be identified as shown at 518. Alternatively, at 520, when there is not sufficient evidence for a variant at the position, it can be determined if there is sufficient evidence for the read matching the reference sequence at the position. When there is sufficient evidence for the read matching the reference at the position, the position can be identified as having no variant, as shown at 522. Alternatively, at 524, when there is not sufficient evidence for the read matching the reference at the position, a no call can be made for the position, indicating there is not sufficient evidence to identify the position as either the reference or a variant.

In various embodiments, a read score can be calculated according to Equation 1 where nf_{deletion} is the number of flow added to represent the reference, nfᵢₙₛₑᵣₜᵢₒₙ is the number of non-empty flows that have no reference, d_{flows} is the sum of the square distances between the read and reference as defined by Equation 2, and coef_{deletion}, coefᵢₙₛₑᵣₜᵢₒₙ, and coef_{intensity} are predefined coefficients. By way of an example, the coef_{deletion} can be in a range of about 1 to about 100, such as for example about 10,coefᵢₙₛₑᵣₜᵢₒₙ can be in a range of about 1 to about 100, such as for example about 10, and coef_{intensity} can be in a range of about 0 to about 10, such as for example about 0.1. Further, a variant score can be calculated according to Equation 5 using the per-read, per-variant score for each read spanning the variant.

$${Score}_{seq . dev} = 1 + ( {coef}_{deletion} \times {nf}_{deletion} ) + \frac{{coef}_{insertion} \times {nf}_{insertion}}{4} + ( {coef}_{intensity} \times {d}_{flows} ) \qquad Equation 1$$

$${d}_{flows} = \sum {( {inten}_{read} - {inten}_{ref} )}^{2} \qquad Equation 2$$

$${Score}_{Variant} = average ( {Scores}_{seq . dev} ) \times log ( \vert Scores \vert ) \qquad Equation 3$$

In various embodiments, a read score can be determined by calculating a Bayesian posterior probability score can be calculated on a per-read, per-variant bases. For each read, P(r|H0) and P(r|H1) can be calculated where H0 is the null hypothesis that there is no variant at a position, and H1 is the predicted variant. The calculation can model the sequence error since the true reference for the read is known. Additionally, the calculation can utilize the reference context surrounding the position and the neighboring flow signals. Log(P(r|H0))−log(P(r|H1)) can estimate the log likelihood that read r is actually sequenced from H1. An average error rate can be determined by taking the sum of the log likelihood of the reads spanning the position. The expected number of reads that may support the hypothesis H1 can be calculated from the average error rate. From the actual number of reads supporting hypothesis H1, the likelihood of the variant at the position can be estimated based on a Poisson distribution.

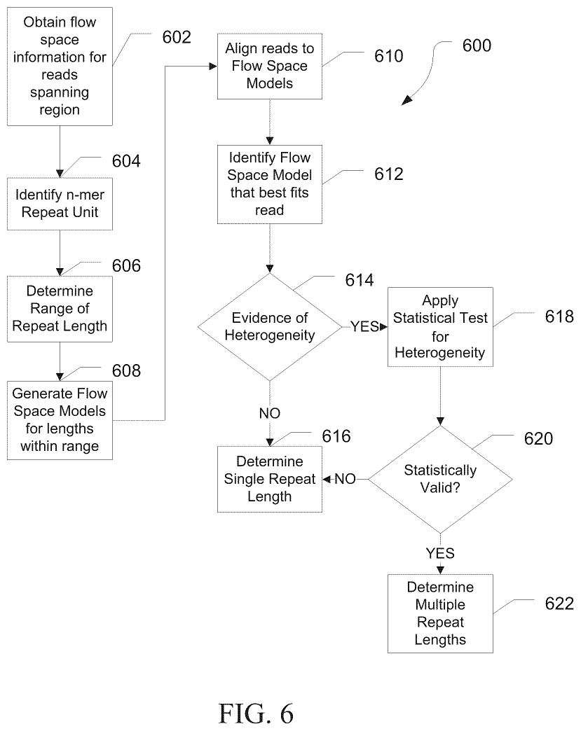

FIG. 6 is an exemplary flow diagram showing a method 600 for identifying indels in di-nucleotide and tri-nucleotide repeat regions. Generally, insertions and deletions in di-nucleotide and tri-nucleotide repeat regions occur by insertion or deletion of entire repeat units, rather than by insertion or deletion of single nucleotides within a repeat unit.

At 602, flow space information for reads spanning a di-nucleotide or tri-nucleotide repeat region can be obtained. At 604, the di-nucleotide or tri-nucleotide repeat unit can be determined. The repeat unit can be the two or three nucleotide sequence that is repeated in the di-nucleotide or tri-nucleotide repeat.

At 606, a range of repeat lengths can be determined. In various embodiments, the range of repeat lengths can be chosen to include the number of repeats in the reference sequence as well as the possible repeat lengths from the reads. For example, when the reference sequence has a repeat length of five, one read has an apparent deletion of a single nucleotide suggesting a repeat length of four, and one read has an apparent insertion of a single nucleotide suggesting a repeat length of six, a range of repeat lengths can be chosen to include four through six. In various embodiments, the range may be chosen to be broader than the suggested repeat lengths, such as in the example a range of three to seven or broader.

At 608, flow space models can be generated for the repeat lengths within the range, and, at 610, the reads can be aligned to the flow space models.

At 612, a flow space model that best fits the flow space information for the read can be identified for the reads that span the di-nucleotide or tri-nucleotide repeat region. Based on the best fit models for the reads, a distribution of apparent repeat lengths can be identified.

At 614, it can be determined if the is evidence of heterogeneity in the repeat length. For example, evidence of heterogeneity may be present when the two observed repeat lengths with the highest number of supporting reads have substantially equal number of supporting reads. In another example, a multi-modal distribution of repeat lengths can indicate the presence of multiple alleles having different repeat lengths in the sample, and the repeat lengths can be determined based upon the modes. At 616, when there is not evidence of heterogeneity, the reported repeat length can be determined, such as based on the observed repeat length with the highest number of supporting reads.

Alternatively, when there is evidence of heterogeneity, at 618, a statistical test can be applied to determine heterogeneity. For example, a Bayesian analysis can be performed to compare the likelihood that the distribution of observed repeat lengths is due to a heterogeneous sample versus due to error.

At 620, a determination can be made to the statistical validity of the hypothesis that the distribution is the result of a heterogeneous sample. When the statistical test supports heterogeneity, multiple reported repeat lengths can be determined based on the distribution of the observed repeat lengths, as illustrate at 622. Alternatively, when the statistical test is inconclusive or does not support heterogeneity, a single repeat length can be reported at 616.

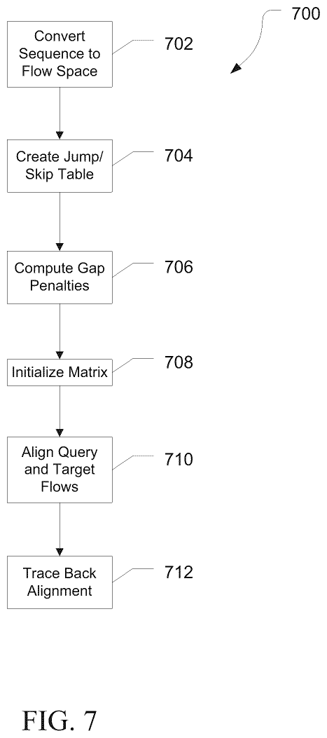

FIG. 7 is an exemplary flow diagram showing a method 700 for aligning reads in flow space.

At 702, a target base sequence can be converted into flow space. The target base sequence can be a portion of the reference sequence to which a read has been mapped. For example, a flow signal vector and a target flow order can be generated from the target base sequence. The target flow order can represent the identities of each base of the target base sequence in order. Collapsing the repeating bases into a single identity with the flow signal vector can represent the number of times a base is repeated.

At 704, a jump/skip table can be created for the query flow order. The query flow order can be the flow order used by the sequencing instrument when generating the reads. The jump/skip table can represent the number of bases to reach the next index within the query flow order with the same base, such as the number of flows that need to be jumped or skipped. Similarly, a reverse jump/skip table can be calculated for the reverse direction.

At 706, gap penalties can be pre-computed. The gap penalty can be based on the reverse jump/skip table.

At 708, the dynamic programming matrix can be initialized. The dynamic programming matrix can be initialized such that a cell within the matrix stores a match score and match traceback, an insertion score and insertion traceback, a deletion score and deletion traceback. The start cells can be initialized with the phase penalty and pre-computed gap penalties.

At 710, the read flow information can be aligned with the target flow information. A dynamic programming algorithm can loop over the flows in the query flow signal vector and the target flow signal vector. As the dynamic programming algorithm progresses, the possible moves can be considered, a horizontal move, a vertical move, and a diagonal move. The horizontal move corresponds to skipping over a target flow base which would represent a deletion in the read. The information from the previous column and same row can be extended into the current cell and can be weighted by the target flow signal that was skipped. For the vertical move, when the query flow base and the target flow base do not match, the target flow order can be padded with empty flows having a flow signal of 0. Additionally, the previous rows score can be penalized by the query flow signal for the current base. Additionally, the previous matching base in the query flow order can be considered. A phase penalty and a pre-computed gap penalty can be added, the move with the maximum score can be kept and the traceback cell can be annotated appropriately. For the diagonal move can be considered when the query flow base and the target flow base match. The score can be determined from the absolute value of the difference between the query flow signal and the target flow signal.

At 712, the alignment can be determined by tracing back along the highest scoring path. When the path includes a match, the flow signals from the query and target can be pushed into the alignment. When the path includes an insertion, for an empty target flow the query flow signal, the empty target signal, and the query flow base can be added to the alignment. Alternatively, for a phased insertion, the query flow bases, the query flow signals, and the target gaps back to the previous matching query flow base can be added to the alignment. When the path includes a deletion, a query gap, the target flow signal, and the target flow base can be added to the alignment. Once the alignment has been traced back, the alignment can be reversed to obtain the alignment in the forward direction.

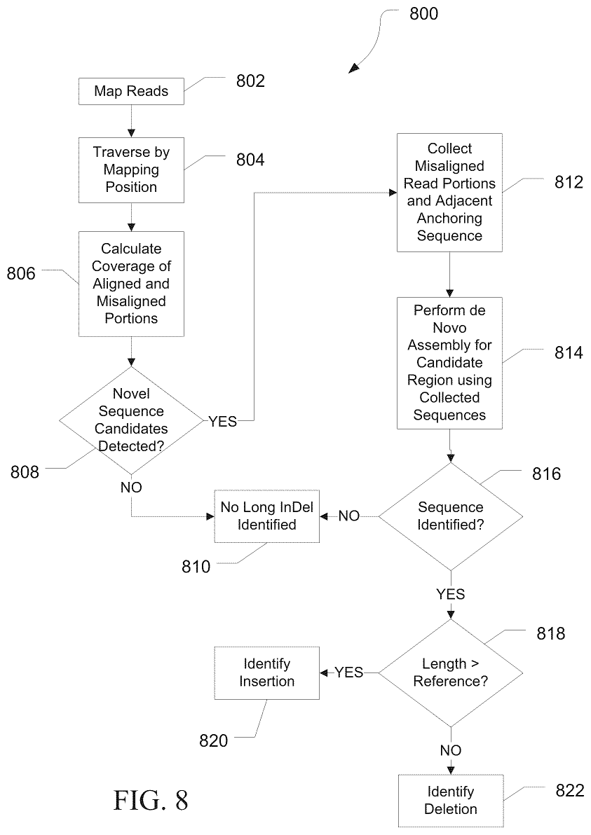

FIG. 8 is an exemplary flow diagram showing a method 800 for identifying insertions or deletions.

At 802, the reads can be mapped to a reference genome.

At 804, the reads can be traversed in order based on the mapping position. In various embodiments, the reads may be placed in a sorted list based on the mapping order, and the list can be traversed.

At 806, the coverage formed by aligned and misaligned portions of reads can be calculated for a genome region. At 808, a determination can be made as to whether a novel sequence candidate is detected. At 810, when no novel sequence candidate is detected, an insertion or deletion is not identified.

Alternatively, when a genome region has a high percentage of misaligned portions, a novel sequence candidate can be detected. In various embodiments, when an insertion or deletion is present, reads of the insertion or deletion can have a mapped portion and an unmapped portion. When multiple reads are present in both directions containing mapped and unmapped portions for the same genome region, the presence of a novel sequence candidate can be detected indicating a potential insertion or deletion.

At 812, misaligned portions of reads and adjacent anchoring sequences can be collected. The adjacent anchoring sequences can be mapped portions of the reads that are adjacent to the potential insertion or deletion.

At 814, de novo assembly can be performed using the misaligned portions of reads and the adjacent anchoring sequences. Various techniques are known in the art to perform de novo assembly. Examples include, but are not limited to, VELVET, SOAPdenovo, ABySS, Forge, etc.

In various embodiments, additional unmapped reads that at least partially overlap with misaligned portions of the partially mapped reads can be included in the local assembly. The inclusion of unmapped reads can allow for the identification of insertions that have a length greater than the read length.

At 816, a determination can be made if the de novo assembly method identified a sequence. For example, the sequence can be identified when the de novo assembly method generates an unambiguous assembly, but may not be identified when the de novo assembly method generates more than one possible assembly or fails to connect the anchoring sequences on either side of the novel sequence candidate region. At 810, when the de novo assembly method does not identify a sequence, an insertion or deletion may not be identified.

Alternatively, at 818, when the de novo assembly method identifies a sequence, comparison can be made between the length of the assembled sequence and the corresponding region in the reference genome. For example, the number of base pairs between the anchoring sequences in the assembled sequence can be compared to the number of base pairs between the anchoring sequences in the reference genome. At 820, when the length of the assembled sequence is greater than the corresponding region of the reference genome, an insertion can be identified in the sample sequence. Alternatively, at 822, when the length of the assembled sequence is not greater than the corresponding region of the reference genome, a deletion can be identified in the sample sequence.

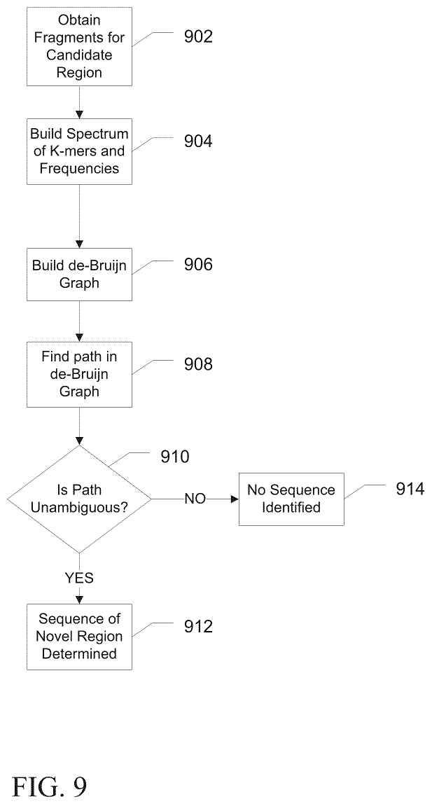

FIG. 9 is an exemplary flow diagram showing a method 900 for performing a local de novo assembly.

At 902, sequence fragments can be obtained for a candidate region. In various embodiments, the sequence fragments can include misaligned portions and corresponding anchor sequences of reads that are partially mapped to the candidate region. Additionally, the sequence fragments can include unmapped reads, such as reads that were unable to be mapped to the reference sequence.

At 904, a spectrum of the fragments can be built. Specifically, a count of possible k-mers (sequences of length k) can be generated from the sequence fragments. For example, with a fragment with a sequence of GATCTAGTA and a k of 4, each of GATC, ATCT, TCTA, CTAG, TAGT, and AGTC occur once. The spectrum can be built using all the fragments obtained for the candidate region. In various embodiments, the fragments in which the k-mers were found can also be tracked.

At 906, a de Bruijn graph can be constructed from the k-mers. For example, a prefix and suffix can be determined for each k-mer. In various embodiments, the prefix can be the starting k-1 bases and the suffix can be the ending k-1 bases. For example, the prefix of TCTA can be TCT with a suffix of CTA. A node in the graph can be assigned for each prefix and suffix, and an edge can be created between a pair of nodes that are the prefix and suffix of a k-mer that is present in within the fragments.

At 908, a path can be found through the graph from the anchoring sequence on one side of the candidate region to the anchoring sequence on the other side of the candidate region. Various techniques are known in the art for finding a path through the graph, such as, for example, the algorithm implemented in Velvet (Genome Res. 2008 May; 18(5):921-829).

At 910, a determination can be made as to whether the determined path is unambiguous. The path can be unambiguous when there are not alternate paths that also satisfy the criteria. For example, two or more paths may exist that start and end at the respective anchoring sequences such that two paths diverge at some point after the starting anchoring sequence and later converge prior to the ending anchoring sequence. In various embodiments, the ambiguous paths may be such that the sequence differs or both the sequence and the sequence length differ.

At 912, when an unambiguous path is identified, a sequence of the candidate region can be determined based on the unambiguous path. Alternatively, at 914, when an unambiguous path is not identified, the sequence of the candidate region may not be determined.

While the present teachings are described in conjunction with various embodiments, it is not intended that the present teachings be limited to such embodiments. On the contrary, the present teachings encompass various alternatives, modifications, and equivalents, as will be appreciated by those of skill in the art.

Further, in describing various embodiments, the specification may have presented a method and/or process as a particular sequence of steps. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the specification should not be construed as limitations on the claims. In addition, the claims directed to the method and/or process should not be limited to the performance of their steps in the order written, and one skilled in the art can readily appreciate that the sequences may be varied and still remain within the spirit and scope of the various embodiments.

The embodiments described herein, can be practiced with other computer system configurations including hand-held devices, microprocessor systems, microprocessor-based or programmable consumer electronics, minicomputers, mainframe computers and the like. The embodiments can also be practiced in distributing computing environments where tasks are performed by remote processing devices that are linked through a network.

It should also be understood that the embodiments described herein can employ various computer-implemented operations involving data stored in computer systems. These operations are those requiring physical manipulation of physical quantities. Usually, though not necessarily, these quantities take the form of electrical or magnetic signals capable of being stored, transferred, combined, compared, and otherwise manipulated. Further, the manipulations performed are often referred to in terms, such as producing, identifying, determining, or comparing.

Any of the operations that form part of the embodiments described herein are useful machine operations. The embodiments, described herein, also relate to a device or an apparatus for performing these operations. The systems and methods described herein can be specially constructed for the required purposes or it may be a general purpose computer selectively activated or configured by a computer program stored in the computer. In particular, various general purpose machines may be used with computer programs written in accordance with the teachings herein, or it may be more convenient to construct a more specialized apparatus to perform the required operations.

Certain embodiments can also be embodied as computer readable code on a computer readable medium. The computer readable medium is any data storage device that can store data, which can thereafter be read by a computer system. Examples of the computer readable medium include hard drives, network attached storage (NAS), read-only memory, random-access memory, CD-ROMs, CD-Rs, CD-RWs, magnetic tapes, and other optical and non-optical data storage devices. The computer readable medium can also be distributed over a network coupled computer systems so that the computer readable code is stored and executed in a distributed fashion.

What is claimed is:

1. A method for identifying sequence variation in a sample, comprising:
receiving, at a processor, a plurality of nucleic acid sequence reads corresponding to the sample;
mapping the plurality of nucleic acid sequence reads to a reference genome;
identifying an n-mer repeat region in the nucleic acid sequence read based on the presence of an n-mer repeat within the reference genome, wherein the n-mer repeat region includes a set of adjacent n-mers;
identifying a repeat unit for the n-mer repeat region, wherein the repeat unit includes a minimum n-mer repeat sequence;
determining a range of repeat lengths inclusive of the repeat lengths in the reference genome and the repeat lengths present in the nucleic acid sequence reads spanning the n-mer repeat region, wherein the repeat length is a number of repeats in the set of adjacent n-mers;
generating a flow space model for each of the repeat lengths within the range of repeat lengths;
aligning flow space information for each nucleic acid sequence read spanning the n-mer repeat region to the flow space models for the repeat lengths within the range;
determining an apparent repeat length for the nucleic acid sequence read based on the flow space model that best fits the aligned flow space information for the nucleic acid sequence read; and
determining a sample repeat length based on a distribution of the apparent repeat lengths for the nucleic acid sequence reads spanning the n-mer repeat region.

2. The method of claim 1, wherein the n-mer repeat is a di-nucleotide repeat or a tri-nucleotide repeat.

3. The method of claim 1, further comprising determining the flow space model that best fits the aligned flow space information by scoring alignments for the repeat lengths within the range.

4. The method of claim 1, further comprising identifying a repeat length variant based on a comparison of the sample repeat length to a reference repeat length.

5. The method of claim 1, wherein the sample repeat length is determined to be the apparent repeat length with a highest number of supporting reads.

6. The method of claim 1, wherein the determining a sample repeat length further comprises identifying multiple repeat lengths based on a multi-modal distribution of the apparent repeat lengths.

7. The method of claim 1, wherein the plurality of nucleic acid sequence reads are provided by a next generation nucleic acid sequence analysis device communicatively connected with the processor and configured to sequence a plurality of nucleic acid fragments from the sample to obtain the plurality of nucleic acid sequence reads.

8. A computer implemented method for identifying sequence variation in a sample, comprising:
receiving a plurality of nucleic acid sequence reads;
aligning the nucleic acid sequence reads to a reference genome to generate aligned portions and misaligned portions of the nucleic acid sequence reads;
detecting a sequence candidate region based on the misaligned portions of the nucleic acid sequence reads;
collecting the misaligned portions and adjacent anchoring sequences for nucleic acid sequence reads within the sequence candidate region;
building a graph of the misaligned portions of the nucleic acid sequence reads to cross the sequence candidate region;
determining an unambiguous path within the graph spanning the sequence candidate region; and
generating an assembled sequence of the sequence candidate region based at least in part on the unambiguous path.

9. The method of claim 8 wherein the sequence candidate region is a soft clipped region where the nucleic acid sequence reads spanning the variant are partially aligned to the reference genome adjacent to the soft clipped region and partially misaligned.

10. The method of claim 9 wherein a first portion of the nucleic acid sequence reads are partially aligned to the left of the soft clipped region and a second portion of the nucleic acid sequence reads are partially aligned to the right of the soft clipped region.

11. The method of claim 8 wherein the sequence candidate region is a noisy region where the nucleic acid sequence reads provide evidence for a large number of potential variants

12. The method of claim 8 wherein the sequence candidate region includes an insertion or a deletion.

13. The method of claim 8 further comprising comparing a length of the assembled sequence and a length of a corresponding region of the reference genome to identify an insertion or a deletion.

14. The method of claim 8, wherein the plurality of nucleic acid sequence reads are provided by a next generation nucleic acid sequence analysis device configured to sequence a plurality of nucleic acid fragments from the sample to obtain the plurality of nucleic acid sequence reads.

15. A system for identifying sequence variation in a sample, comprising:
a processor in communication with a next generation nucleic acid sequence analysis device, the processor configured to:
receive a plurality of nucleic acid sequence reads;
map the plurality of nucleic acid sequence reads to a reference genome;
identify an n-mer repeat region in the nucleic acid sequence read based on the presence of an n-mer repeat within the reference genome, wherein the n-mer repeat region includes a set of adjacent n-mers;
identify a repeat unit for the n-mer repeat region, wherein the repeat unit includes a minimum n-mer repeat sequence;
determine a range of repeat lengths inclusive of the repeat lengths in the reference genome and the repeat lengths present in the nucleic acid sequence reads spanning the n-mer repeat region, wherein the repeat length is a number of repeats in the set of adjacent n-mers;
generating a flow space model for each of the repeat lengths within the range of repeat lengths;
align flow space information for each nucleic acid sequence read spanning the n-mer repeat region to the flow space models for repeat lengths within the range;
determine an apparent repeat length for the nucleic acid sequence read based on the flow space model that best fits the aligned flow space information for the nucleic acid sequence read; and
determine a sample repeat length based on a distribution of the apparent repeat lengths for the nucleic acid sequence reads spanning the n-mer repeat region.

16. The system of claim 15, wherein the n-mer repeat is a di-nucleotide repeat or a tri-nucleotide repeat.

17. The system of claim 15, wherein the flow space model that best fits the aligned flow space information is determined by scoring alignments for the repeat lengths within the range.

18. The system of claim 15, wherein the sample repeat length is determined to be the apparent repeat length with a highest number of supporting reads.

19. The system of claim 15, wherein the processor is further configured to identify multiple repeat lengths based on a multi-modal distribution of the apparent repeat lengths.

20. The system of claim 15, wherein the processor is configured to identify a repeat length variant based on a comparison of the sample repeat length to a reference repeat length.
